(19) United States
(12) Patent Application Publication
(10) Pub. No.: US 2013/0164247 A1
(43) Pub. Date: Jun. 27, 2013

(54) TRANSPARENT MICROEMULSIONS WITH A FILM-FORMING STYLING POLYMER AND METHODS FOR MAKING THE SAME

(71) Applicant: Jisook Baek, Phoenix, AZ (US)

(72) Inventor: Jisook Baek, Phoenix, AZ (US)

(21) Appl. No.: 13/336,203

(22) Filed: Dec. 23, 2011

(51) Int. Cl. A61K 8/81; A61Q 5/06

(52) U.S. Cl. 424/70.15

(57) ABSTRACT

Transparent microemulsions and methods for making the same are provided. In accordance with an exemplary embodiment, a transparent microemulsion comprises an aqueous phase, an oil phase, an emulsifier, and vinyl caprolactom/vinyl pyrrolidone/dimethyl-aminoethyl methacrylate copolymer. An exemplary method for forming a transparent microemulsion hair styling product comprises forming an oil phase comprising a lipophilic ingredient, forming an aqueous phase, providing an emulsifier, and combining the oil phase, the aqueous phase, and the emulsifier to form a mixture. The mixture is combined with vinyl caprolactom/vinyl pyrrolidone/dimethylaminoethyl methacrylate copolymer.

### TECHNICAL FIELD

The present invention generally relates to microemulsions and methods for making the same, and more particularly relates to transparent microemulsions with a film-forming styling polymer and methods for making the same.

### BACKGROUND

Microemulsions are optically transparent, isotropic, thermodynamically stable ternary systems of an aqueous liquid phase and an oil phase stabilized by an interfacial film of an emulsifier. Microemulsions are monodispersed spherical droplets, with a diameter less than 100 nm, of water in oil or oil in water, depending on the nature of the emulsifier. Microemulsions are in contrast to macroemulsions, which are kinetically stable, have droplets with a diameter in the range of about 1 to 10 microns and are thus opaque, and have low surface area in the range of about 15 square meters per gram (m²/g) compared to the high surface area of microemulsions (about 200 m²/g). In addition, macroemulsions are shear sensitive during mixing while microemulsions are not. Frequently, microemulsions are in gel form and exhibit a vibrating or “ringing effect” when tapped.

Microemulsions have been used as hair styling products but could be improved by the addition of a film-forming styling polymer or polymers. Film-forming styling polymers can add a variety of beneficial characteristics, such as stiffness, flexibility, shine and the like, to the hair depending on the film-forming styling polymer in the microemulsion. Generally, however, the incorporation of film-forming styling polymers within microemulsions has not been successful because the film-forming styling polymers tend to turn microemulsions hazy. This haziness conveys a quality of instability of the microemulsion to users who are used to transparent hair gels. While manufacturers often try to hide the haziness by packaging the microemulsion hair products in dark jars or other packaging or by adding colorants or dye to the hair products, there is still a need for a transparent microemulsion hair product comprising a film-forming styling polymer.

Accordingly, it is desirable to provide a transparent microemulsion comprising a film-forming styling polymer. In addition, it is desirable to provide a transparent microemulsion comprising vinyl caprolactom/vinyl pyrrolidone/dimethylaminoethyl methacrylate copolymer. It is also desirable to provide a method for making a transparent microemulsion comprising vinyl caprolactom/vinyl pyrrolidone/dimethylaminoethyl methacrylate copolymer. Furthermore, other desirable features and characteristics of the present invention will become apparent from the subsequent detailed description of the invention and the appended claims, taken in conjunction with the accompanying drawings and this background of the invention.

### BRIEF SUMMARY

Transparent microemulsions and methods for making the same are provided. In accordance with an exemplary embodiment, a transparent microemulsion comprises an aqueous phase, an oil phase, an emulsifier, and vinyl caprolactom/vinyl pyrrolidone/dimethyl-aminoethyl methacrylate copolymer.

In accordance with another exemplary embodiment, a method for forming a transparent microemulsion hair styling product comprises forming an oil phase comprising a lipophilic ingredient, forming an aqueous phase, providing an emulsifier, and combining the oil phase, the aqueous phase, and the emulsifier to form a mixture. The mixture is combined with vinyl caprolactom/vinyl pyrrolidone/dimethylaminoethyl methacrylate copolymer.

In accordance with a further exemplary embodiment, a transparent microemulsion hair styling product comprises about 20 to about 60 wt. % aqueous phase, about 5 to about 25 wt. % oil phase comprising an emollient, about 20 to about 40 wt. % emulsifiers, and about 0.15 to about 0.6 wt. % (active level) vinyl caprolactom/vinyl pyrrolidone/dimethylaminoethyl methacrylate copolymer.

### BRIEF DESCRIPTION OF THE DRAWINGS

The present invention will hereinafter be described in conjunction with the following drawing figures, wherein like numerals denote like elements, and

FIG. 1 is an illustration of the molecular structure of vinyl caprolactom/vinyl pyrrolidone/dimethylaminoethyl methacrylate copolymer;

FIG. 2 is an illustration of the molecular structure of polyvinylpyrrolidone;

FIG. 3 is an illustration of the molecular structure of vinylpyrrolidone/vinyl acetate copolymer; and

FIG. 4 is an illustration of vinylpyrrolidone/dimethylaminopropyl-acrylamide copolymer.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Various embodiments of the transparent oil-in-water microemulsions contemplated herein comprise an aqueous phase, an oil phase, an emulsifier, and the film-forming styling polymer vinyl caprolactom/vinyl pyrrolidone/dimethylaminoethyl methacrylate copolymer. The inventor has found that vinyl caprolactom/vinyl pyrrolidone/dimethylaminoethyl methacrylate copolymer can be used to enhance the styling properties of a microemulsion hair styling product without decreasing the transparency of the microemulsion. As used herein, the term “transparent” or “transparency” means that the microemulsion appears optically clear to the naked eye and/or that the microemulsion has the property of transmitting rays of light therethrough so that bodies situated beyond or behind it can be distinctly seen.

The transparent microemulsions contemplated herein comprise an aqueous phase. Water is present in an amount of about 20 to about 60 weight percent (wt. %) of the total weight of the transparent microemulsion, for example, about 40 wt. % of the total weight of the transparent microemulsion. In an exemplary embodiment, the water is deionized water. In addition to water, the aqueous phase contains one or more cosmetically compatible, hydrophilic co-solvents. The amount and species of the co-solvents are selected according to their properties and their compatibility with other components in the microemulsion. In an embodiment, the co-solvents are monohydric or polyhydric alcohols that are soluble in the aqueous phase and are liquid at room temperature. In one embodiment, the alcohol is present in an amount of from 0 to about 20 wt. %, for example, from about 1 to about 10 wt. %, of the total microemulsion. Alcohols can be those conventionally used for cosmetic purposes, for example, monohydric C1 to C6 alcohols such as ethanol and isopropanol and polyhydric C2 to C6 alcohols such as polyethylene glycol, glycerol, and sorbitol. As used herein, the term CX refers to the number X of carbons in the molecule, e.g., alcohol. Buffers and other pH adjusting agents can be included to achieve or stabilize the desirable pH.

In addition to the aqueous phase, the transparent microemulsion comprises an oil phase. In an exemplary embodiment, the oil phase of the transparent microemulsions contemplated herein is present in an amount of about 5 to about 25 wt. % of the total weight of the micro emulsion. The oil phase comprises ingredients that are liquid at room temperature (about 25° C.) and other lipophilic and additive ingredients. In an embodiment, the oil phase does not contain waxes that are solid at room temperature or these waxes are present in an amount that does not decrease the optical transparency of the microemulsion, for example, in amounts less than 5 wt. % of the total weight of the transparent microemulsion.

Oils and lipophilic materials suitable for use in the oil phase include, but are not limited to, animal oils, mineral oils, plant oils, silicon oils, hydrocarbon oils, hydrogenated polyolefins, liquid alcohols with at least 8 carbon atoms, especially branched alcohols, oils from fatty acids and polyols, oils from fatty acids and monohydric C1 to C30 alcohols and hydrophobic waxes, and mixtures thereof. Examples of suitable oils include cycloparaffins, paraffin oils, polydecene, mineral oils, isohexadecane, dodecane, isoeicosane, liquid polydimethylsiloxane, cyclotetrasiloxane, cyclopentasiloxane, phenyltrimethicone, isocetyl palmitate, isopropyl myristate, isopropyl palmitate, isopropyl stearate, octyl isostearate, octylcocoate, octyl palmitate, octyl dodecyl myristate, caprylic/capric triglyceride, butyl octanol, hexyl octanol, butyl decanol, hexyl decanol, octyl dodecanol, hexyl decanol, stearyl heptanoate, isohexyl decanoate, isodecyl octanoate, dibutyl adipate, dicarpylyl ether, C12 to C15 alkyl benzoate, hydrogenated polyisobutene, squalane, squalene, native oils, such as jojoba oil, olive oil, sun flower seed oil, soy bean oil, peanut oil, colza oil, almond oil, palm oil, coconut oil, castor oil, wheat germ oil, grape seed oil, thistle oil, candle oil macadamia nut oil, corn seed oil, avocado oil, and the like.

The transparent microemulsions contemplated herein further comprise one or more emulsifiers to promote dispersion of the water phase in the oily phase of the transparent oil-in-water microemulsion contemplated herein. The emulsifiers selected for use in the microemulsions contemplated herein and the amounts of the emulsifiers are based on the properties of the emulsifiers and the components and amounts of the components of the oil phase. The emulsifiers are present in an amount of from about 20 to about 40 wt. % of the total weight of the transparent microemulsion.

The emulsifier comprises any conventional emulsifier known to promote dispersion between an aqueous phase and an oil phase to form a transparent microemulsion. Such emulsifiers include nonionic, anionic, amphoteric, and zwitterionic emulsifiers and combinations thereof In an exemplary embodiment, the emulsifier is a nonionic emulsifier. Examples of nonionic emulsifiers include, but are not limited to:

ethoxylated fatty alcohols, fatty acids, fatty acid glycerides or alkylphenols, in particular addition products of from 2 to 30 mol of ethylene oxide and/or 1 to 5 mol of propylene oxide onto C8 to C22 fatty alcohols, onto C12 to C22 fatty acids or onto alkyl phenols having 8 to 15 carbon atoms in the alkyl group,

C12 to C22 fatty acid mono- and diesters of addition products of from 1 to 30 mol of ethylene oxide onto glycerol,

addition products of from 5 to 60 mol of ethylene oxide onto castor oil or onto hydrogenated castor oil,

fatty acid sugar esters, in particular esters of sucrose and one or two C8 to C22 fatty acids, such as, sucrose cocoate, sucrose dilaurate, sucrose distearate, sucrose laurate, sucrose myristate, sucrose oleate, sucrose palmitate, sucrose ricinoleate, sucrose stearate,

esters of sorbitan and one, two or three C8 to C22 fatty acids and a degree of ethoxylation of from 4 to 20,

polyglyceryl fatty acid esters, in particular of one, two or more C8 to C22 fatty acids and polyglycerol having preferably 2 to 20 glyceryl units, and

alkyl glucosides, alkyl oligoglucosides and alkyl polyglucosides having C8 to C22 alkyl groups, for example, decylglucoside or laurylglucoside.

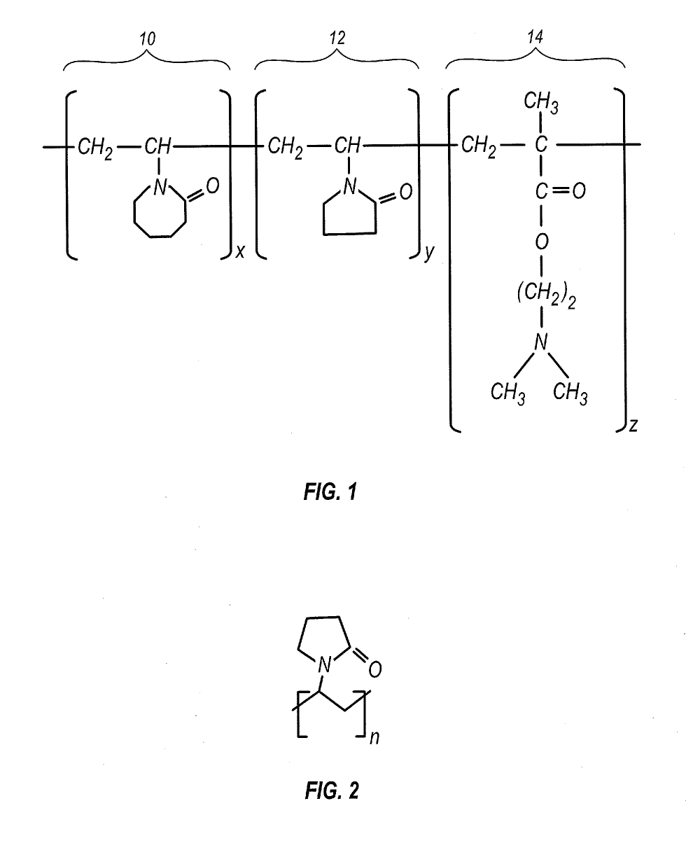

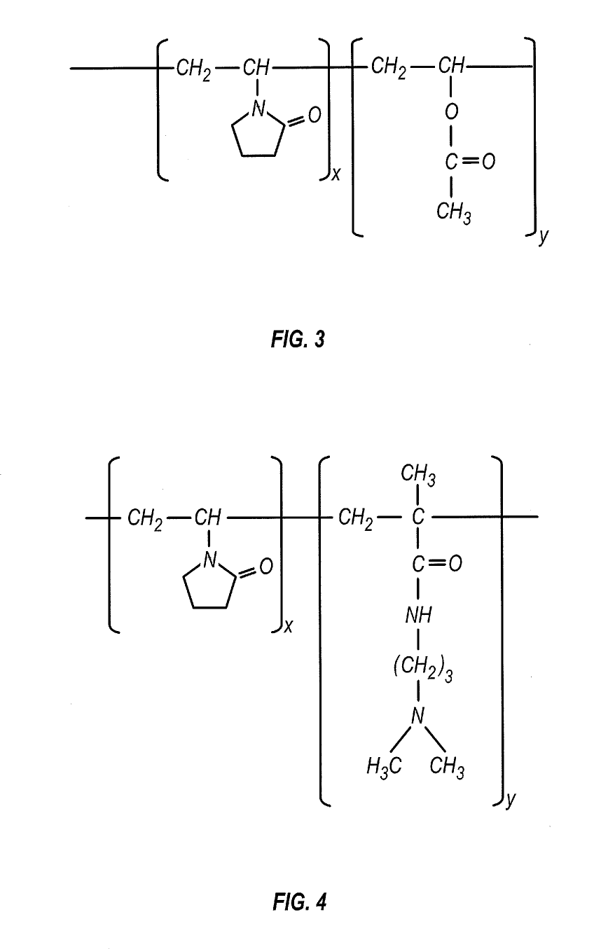

As noted above, the transparent microemulsions contemplated herein further comprise the film-forming copolymer vinyl caprolactom/vinyl pyrrolidone/dimethylaminoethyl methacrylate. Vinyl caprolactom/vinyl pyrrolidone/dimethylaminoethyl methacrylate copolymer has the structure illustrated in FIG. 1, wherein the first polymer linkage 10 is that of vinyl caprolactom, the second polymer linkage 12 is that of vinyl pyrrolidone, and the third polymer linkage 14 is that of dimethylaminoethyl methacrylate. In an exemplary embodiment, the vinyl caprolactom/vinyl pyrrolidone/dimethylaminoethyl methacrylate copolymer is present in the transparent microemulsion in an amount no greater than 0.6 wt. % (active level) of the total weight of the transparent microemulsion. As used herein, the term “active level” means the amount of the copolymer alone, excluding any water or other solvents or additives. In another embodiment, the vinyl caprolactom/vinyl pyrrolidone/dimethylaminoethyl methacrylate copolymer is present in an amount of about 0.15 to about 0.6 wt. %, for example, about 0.45 wt. %, of the total weight of the transparent microemulsion. Without wishing to be bound by theory, it is believed that the vinyl caprolactom/vinyl pyrrolidone/dimethylaminoethyl methacrylate copolymer facilitates integration of the small droplets of the water phase into the oil phase such that the transparency of the microemulsion is maintained. This is in contrast to other similarly-structured film-forming styling polymers, such as polyvinyl pyrrolidone, having the structure illustrated in FIG. 2, polyvinylpyrrolidone/vinyl acetate, having the structure illustrated in FIG. 3, or vinylpyrrolidone/dimethylaminopropyl-acrylamide, having the structure illustrated in FIG. 4, all of which cause microemulsions to turn hazy, as discussed in more detail below. In addition, the vinyl caprolactom/vinyl pyrrolidone/dimethylaminoethyl methacrylate copolymer can provide hair styling properties including shine, strong hold, humidity resistance, and/or water solubility. Accordingly, in an exemplary embodiment, the only film-forming styling polymer or copolymer in the transparent microemulsion contemplated herein is the vinyl caprolactom/vinyl pyrrolidone/dimethylaminoethyl methacrylate copolymer. Vinyl caprolactom/vinyl pyrrolidone/dimethylaminoethyl methacrylate copolymer is available as Advantage® S from Ashland Specialty Ingredients, Wayne, N.J.

The transparent microemulsions contemplated herein may comprise other optional ingredients conventionally used in hair styling products as long as such ingredients do not adversely affect the transparency of the microemulsions. These ingredients may include preservatives, fragrances, stabilizers, sunscreen agents, and the like. In an exemplary embodiment, each of these ingredients is present in the transparent microemulsions contemplated herein in an amount no greater than about 5 wt. % based on the total weight of the transparent microemulsion.

A method for making a transparent microemulsion as contemplated herein includes forming an oil phase, forming an aqueous phase, combining the oil phase, the aqueous phase, and an emulsifier to form a mixture, and combining the mixture with the vinyl caprolactom/vinyl pyrrolidone/dimethylaminoethyl methacrylate copolymer. It will be appreciated that the method is not limited to this order of the steps. For example, the oil phase can be formed before the aqueous phase, the emulsifier can be added to the oil phase before combination with the aqueous phase, and/or the vinyl caprolactom/vinyl pyrrolidone/dimethylaminoethyl methacrylate copolymer can be added to the aqueous phase before the aqueous phase is combined with the oil phase. Heat can also be administered to the components to facilitate the process.

In one exemplary embodiment, the emulsifier or emulsifiers are added to a vessel along with the ingredients of the oil phase and are heated to a temperature that is sufficient to melt the components and form a microemulsion but is not so high as to burn or discolor the oil phase. In an exemplary embodiment, the temperature is in the range of about 82 to about 85° C. Any suitable type of mixing, for example, low turbine and sidesweep agitation, can be used to mix the ingredients. The co-solvent or co-solvents of the aqueous phase is added to another vessel and mixing and heating are administered. The water of the aqueous phase is added to the co-solvent. Mixing continues until the aqueous phase is homogenous. The aqueous phase then is slowly added to the oil phase with mixing. The mixing speed is accelerated as the aqueous phase is added to the oil phase. The viscosity will begin to increase as more water of the aqueous phase is added, taking on a gel-like consistency. Mixing and heat are maintained until the mixture is microscopically homogeneous. Once the mixture is microscopically homogeneous, the temperature is lowered, to a temperature, for example, in the range of about 70 to about 75° C., and the vinyl caprolactom/vinyl pyrrolidone/dimethylaminoethyl methacrylate copolymer is added. Any preservatives, fragrance and optional ingredients also are added at this point. The resulting transparent microemulsion can be cooled further, such as, for example, to a temperature in the range of from 60 to 65° C., and dispensed into packaging, such as jars, tubes, and the like.

The following is an exemplary embodiment of a transparent microemulsion as contemplated herein, with each of the components set forth in weight percent of the transparent microemulsion. The example is provided for illustration purposes only and is not meant to limit the various embodiments of the transparent microemulsion in any way.

### EXAMPLE

| | |
|---|---|
| Ingredient | Wt. % |

Oil Phase:

| | |
|---|---|
| Isopropyl Myristate | 15.00 |

Emulsifiers:

| | |
|---|---|
| Oleth-10 | 8.00 |
| Oleth-20 | 6.00 |
| Ceteareth-20 | 2.00 |
| PEG-7 Glycerol Cocoate | 2.00 |
| PEG-25 Hydrogenated Castor Oil | 16.00 |

Water Phase

| | |
|---|---|
| Propylene Glycol | 7.00 |
| Methylparaben | 0.13 |
| Benzophenone-4 | 0.08 |
| Phenoxyethanol, | 0.30 |
| Methylisothiazolinone | |
| Fragrance | 0.60 |
| Deionized Water | 40.89 |
| Vinyl caprolactom/vinyl | 2.00 (0.6 wt. % |
| pyrrolidone/dimethylaminoethyl | active concentration) |
| methacrylate copolymer | |
| (30% active in solution) | |
| Total | 100.00 |

The transparent microemulsion of the Example was produced by adding to a first vessel the following ingredients in order and mixing well after each addition: oleth-10, oleth-20, ceteareth-20, PEG-7 glycerol cocoate, isopropyl myristate and PEG-25 hydrogenated castor oil. This mixture A was heated to 82-85° C. Care was taken not to heat the mixture above 87° C. so as not to burn or discolor the mixture. The mixture then was subjected to low turbine and sidesweep agitation.

Propylene glycol, methylparaben, and benzophenone were added to a second vessel. Mixing in the vessel was started and the ingredients were heated to 82-85° C. All ingredients were mixed until dissolved and the temperature was maintained at 82-85° C. Next, the deionized water was added to second vessel to form mixture B, which was mixed until homogenous. The vessel lid was closed after the addition of each ingredient to minimize evaporation, particularly of the deionized water.

Mixture B was then added to mixture A with a gradual increase in agitation. Mixture B was slowly added to mixture A, with a gradual increase in the addition rate. The viscosity began to increase as more water in mixture B was added to mixture A and the resulting microemulsion began to take on a gel-like consistency. The temperature was maintained at 82-85° C. and mixing was continued until the microemulsion was microscopically homogeneous. The lid of the vessel was kept closed whenever possible to minimize evaporation. Once homogeneous, the microemulsion was cooled to 73-76° C. and the fragrance, phenoxyethanol, methylisothiazolinone, and the vinyl caprolactom/vinyl pyrrolidone/dimethylaminoethyl methacrylate copolymer (Advantage S from Ashland Specialty Ingredients) were added. The microemulsion was mixed well after each addition. The transparent microemulsion then was cooled to 60-65° C. and poured into jars. The resulting microemulsion of this EXAMPLE was a clear/transparent gel with no visible signs of haziness.

The transparent microemulsion of this EXAMPLE was compared to similar microemulsions made with other similarly-structured hair styling polymers. A COMPARISON MICROEMULSION 1 was made using the same method and formulation above with the exception that 0.6 wt. % (active level) polyvinylpyrrolidone was substituted for the 0.6 wt. % (active level) vinyl caprolactom/vinyl pyrrolidone/dimethylaminoethyl methacrylate copolymer. The resulting COMPARISON MICROEMULSION 1 was hazy.

A COMPARISON MICROEMULSION 2 was made using the same method and formulation as used for the EXAMPLE transparent microemulsion with the exception that 0.6 wt. % (active level) vinylpyrrolidone/vinyl acetate copolymer was substituted for the 0.6 wt. % (active level) vinyl caprolactom/vinyl pyrrolidone/dimethylaminoethyl methacrylate copolymer. The resulting COMPARISON MICROEMULSION 2 was hazy.

A COMPARISON MICROEMULSION 3 was made using the same method and formulation as used for the EXAMPLE transparent microemulsion with the exception that 0.2 wt. % (active level) vinylpyrrolidone/dimethylaminopropyl-acrylamide copolymer was substituted for the 0.6 wt. % (active level) vinyl caprolactom/vinyl pyrrolidone/dimethylaminoethyl methacrylate copolymer. Again, the COMPARISON MICROEMULSION 3 was hazy, even with only 0.2 wt. % (active level) vinylpyrrolidone/dimethylaminopropyl-acrylamide copolymer.

Accordingly, various embodiments of the transparent microemulsions contemplated herein comprise an aqueous phase, an oil phase, an emulsifier, and vinyl caprolactom/vinyl pyrrolidone/dimethylaminoethyl methacrylate copolymer. The inventor has found that vinyl caprolactom/vinyl pyrrolidone/dimethylaminoethyl methacrylate copolymer can be used to enhance the styling properties of a microemulsion without decreasing the transparency of the microemulsion. While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A transparent microemulsion comprising:
an aqueous phase;
an oil phase;
an emulsifier; and
vinyl caprolactom/vinyl pyrrolidone/dimethylaminoethyl methacrylate copolymer.

2. The transparent microemulsion of claim 1, wherein the aqueous phase comprises about 20 to about 60 wt. % of a total weight of the transparent microemulsion.

3. The transparent microemulsion of claim 1, wherein the aqueous phase comprises deionized water.

4. The transparent microemulsion of claim 1, wherein the aqueous phase comprises co-solvents in an amount of about 1 to about 10 wt. % of a total weight of the transparent microemulsion.

5. The transparent microemulsion of claim 4, wherein the co-solvents are chosen from a group comprising monohydric C1 to C6 alcohols and polyhydric C2 to C6 alcohols.

6. The transparent microemulsion of claim 1, wherein the oil phase is present in an amount of about 5 to about 25 wt. % of a total weight of the transparent microemulsion.

7. The transparent microemulsion of claim 1, wherein the emulsifier is nonionic.

8. The transparent microemulsion of claim 1, wherein the emulsifier is present in an amount of about 20 to about 40 of a total weight of the transparent microemulsion.

9. The transparent microemulsion of claim 1, wherein the vinyl caprolactom/vinyl pyrrolidone/dimethylaminoethyl methacrylate copolymer is present in an amount in a range of about 0.15 to about 0.6 wt. % (active level) of a total weight of the transparent microemulsion.

10. The transparent microemulsion of claim 9, wherein the vinyl caprolactom/vinyl pyrrolidone/dimethylaminoethyl methacrylate copolymer is present in an amount of about 0.45 wt. % (active level) of a total weight of the transparent microemulsion.

11. A method for forming a transparent microemulsion hair styling product, the method comprising the steps of:
forming an oil phase comprising a lipophilic ingredient;
forming an aqueous phase;
providing an emulsifier;
combining the oil phase, the aqueous phase, and the emulsifier to form a mixture;
and
combining the mixture and vinyl caprolactom/vinyl pyrrolidone/dimethylaminoethyl methacrylate copolymer.

12. The method of claim 11, wherein providing the emulsifier comprises combining the emulsifier and the lipophilic ingredient and wherein combining the oil phase, the aqueous phase, and the emulsifier comprises combining the oil phase containing the emulsifier and the aqueous phase.

13. The method of claim 11, wherein forming the aqueous phase is performed before forming the oil phase.

14. The method of claim 11, wherein forming the oil phase comprises mixing the oil phase and increasing a temperature of the oil phase above room temperature.

15. The method of claim 11, wherein forming the aqueous phase comprises mixing the aqueous phase and increasing a temperature of the aqueous phase above room temperature.

16. The method of claim 11, wherein combining the oil phase, the aqueous phase, and the emulsifier comprises adding the aqueous phase to the oil phase.

17. A transparent microemulsion hair styling product comprising:
about 20 to about 60 wt. % aqueous phase;
about 5 to about 25 wt. % oil phase comprising an emollient;
about 20 to about 40 wt. % emulsifiers; and
about 0.15 to about 0.6 wt. % (active level) vinyl caprolactom/vinyl pyrrolidone/dimethylaminoethyl methacrylate copolymer.

18. The transparent microemulsion hair styling product of claim 17, wherein the emulsifiers comprise oleth-10, oleth-20, ceteareth-20, PEG-7 glycerol cocoate, and PEG-25 hydrogenated castor oil.

19. The transparent microemulsion hair styling product of claim 17, wherein the emollient is isopropyl myristate.

20. The transparent microemulsion hair styling product of claim 17, wherein the aqueous phase comprises propylene glycol.
